(19) United States
(12) Patent Application Publication
(10) Pub. No.: US 2023/0211074 A1
(43) Pub. Date: Jul. 6, 2023

(54) INTRAVENOUS PRESSURE ASSEMBLY

(71) Applicant: CareFusion 303, Inc., San Diego, CA (US)

(72) Inventors: Kelly Kloster HON, Del Mar, CA (US); Gilbert LEUNG, La Verne, CA (US)

(21) Appl. No.: 18/078,705

(22) Filed: Dec. 9, 2022

Related U.S. Application Data
(60) Provisional application No. 63/296,021, filed on Jan. 3, 2022.

(51) Int. Cl. A61M 5/142; A61M 5/168; A61M 39/10

(52) U.S. Cl. CPC A61M 5/1424; A61M 5/16804; A61M 39/10; A61M 2205/586

(57) ABSTRACT

An intravenous IV pressure assembly includes a body, an outlet port disposed on the body and a connector coupled to the outlet port, the connector being configured to couple with a fluid reservoir and the IV pressure assembly configured as a pressurized container. IV sets with an IV pressure assembly and methods of operating an IV pressure assembly are also provided.

### CROSS-REFERENCE TO RELATED APPLICATIONS

This application claims the benefit of priority under 35 U.S.C. § 119 to U.S. Provisional Patent Application Ser. No. 63/296,021, entitled “INTRAVENOUS PRESSURE ASSEMBLY,” filed on Jan. 3, 2022, the entirety of which is incorporated herein by reference.

### TECHNICAL FIELD

The present disclosure generally relates to intravenous (IV) sets, in particular to pressurized devices for IV sets.

### BACKGROUND

Medical treatments often include the infusion of a medical fluid (e.g., blood, plasma, saline) to patients using an IV catheter that is connected though an arrangement of flexible tubing and fittings, commonly referred to as an “IV set,” to a source of fluid, for example, an IV blood bag. During operation, medical fluid may be required quickly at greatly increased flow rates as shorter times to blood transfusions have been associated with decreased death risk in trauma patients. Typical IV sets use a cylindrical hand pump that is squeezed repeatedly by hand to rapidly increase fluid flow rate, resulting in muscle fatigue. The geometry of the cylindrical hand pump limits the amount of fluid and how quickly it takes for new fluid from the fluid source to pass into the pump chamber as the pump chamber returns to the cylindrical shape after being squeezed.

For these reasons, it is desirable to provide an IV set with a pressurized device to reduce hand muscle fatigue, to increase the volume of fluid received into the hand pump after being squeezed and to decrease the time to fill the hand pump after being squeezed.

### SUMMARY

In some embodiments of the disclosure, an intravenous IV pressure assembly includes a body, an outlet port disposed on the body and a connector coupled to the outlet port, the connector configured to couple with a fluid reservoir, wherein the IV pressure assembly is configured as a pressurized container.

In some embodiments of the disclosure, an IV system includes an IV set having one or more IV components and one or more IV tubes, and an IV pressure assembly, including a body, an outlet port disposed on the body and a connector coupled to the outlet port, the connector configured to couple with a fluid reservoir, wherein the IV pressure assembly is a pressurized container configured to force fluid within the fluid reservoir into the IV set at a fluid flow rate exceeding one of a maximum gravity based fluid flow rate and a maximum infusion pump fluid flow rate.

In some embodiments of the disclosure, a method of operating an IV pressure assembly includes coupling the IV pressure assembly to a fluid reservoir, the IV pressure assembly comprising: a body comprising a pressurized container; an outlet port disposed on the body; and a connector coupled to the outlet port, the connector configured to couple with the fluid reservoir. The method also includes opening a fluid flow pathway between the IV pressure assembly and the fluid reservoir; providing pressure into the fluid reservoir through the fluid flow pathway; and ceasing providing pressure into the fluid reservoir by one of: depleting a pressurized fluid in the pressurized container; uncoupling the connector from the fluid reservoir; adjusting a valve of the IV pressure assembly to a closed position; and placing a seal into a blocking position within the fluid flow pathway.

The foregoing and other features, aspects and advantages of the disclosed embodiments will become more apparent from the following detailed description and accompanying drawings.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example patient care system having four fluid infusion pumps, each of which is connected to a respective fluid supply for pumping the contents of the fluid supply to a patient.

FIG. 2 depicts a top view of a typical assembled IV infusion set with a hand pump.

FIG. 3 depicts a front view of a pressure assembly and fluid reservoir, according to aspects of the disclosure.

FIG. 4 depicts a perspective view of an IV set with the pressure assembly and fluid reservoir of FIG. 3, according to aspects of the disclosure.

FIG. 5 depicts a perspective view of an IV set and the pressure assembly of FIG. 3 connecting an infusion pump to a patient, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

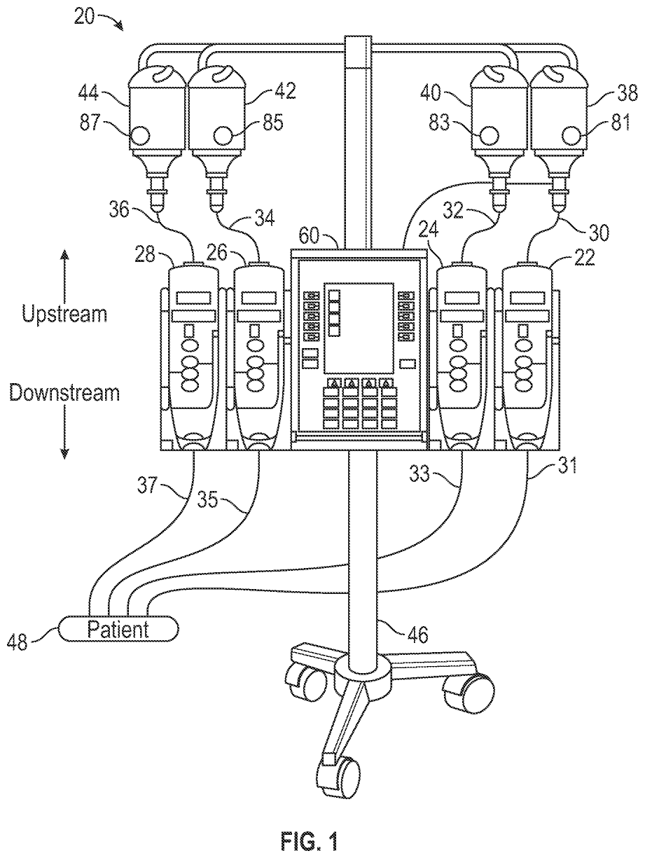

Referring now in more detail to the drawings in which like reference numerals refer to like or corresponding elements among the several views, there is shown in FIG. 1 a patient care system 20 having four infusion pumps 22, 24, 26, and 28 each of which is fluidly connected with an upstream fluid line 30, 32, 34, and 36, respectively. Each of the four infusion pumps 22, 24, 26, and 28 is also fluidly connected with a downstream fluid line 31, 33, 35, and 37, respectively. The fluid lines can be any type of fluid conduit, such as an IV administration set, through which fluid can flow through. It should be appreciated that any of a variety of pump mechanisms can be used including syringe pumps.

Fluid supplies 38, 40, 42, and 44, which may take various forms but in this case are shown as bottles, are inverted and suspended above the pumps. Fluid supplies may also take the form of bags or other types of containers including syringes. Both the patient care system 20 and the fluid supplies 38, 40, 42, and 44 are mounted to a roller stand, IV pole 46, table top, etc.

A separate infusion pump 22, 24, 26, and 28 is used to infuse each of the fluids of the fluid supplies into the patient. The infusion pumps are flow control devices that will act on the respective fluid line to move the fluid from the fluid supply through the fluid line to the patient 48. Because individual pumps are used, each can be individually set to the pumping or operating parameters required for infusing the particular medical fluid from the respective fluid supply into the patient at the particular rate prescribed for that fluid by the physician. Such medical fluids may include drugs or nutrients or other fluids. The infusion pumps 22, 24, 26, and 28 are controlled by a pump control unit 60.

Fluid supplies 38, 40, 42, and 44 are each coupled to an electronic data tag 81, 83, 85, and 87, respectively, or to an electronic transmitter. Any device or component associated with the infusion system may be equipped with an electronic data tag, reader, or transmitter.

Typical infusion sets may also be gravity sets that do not require use of an infusion pump. For example, any of fluid supplies 38, 40, 42, and 44 may be directly connected to the patient 48 via a gravity IV set, wherein gravity causes the fluid to flow through the infusion set and into the patient 48 without the aid of a pump.

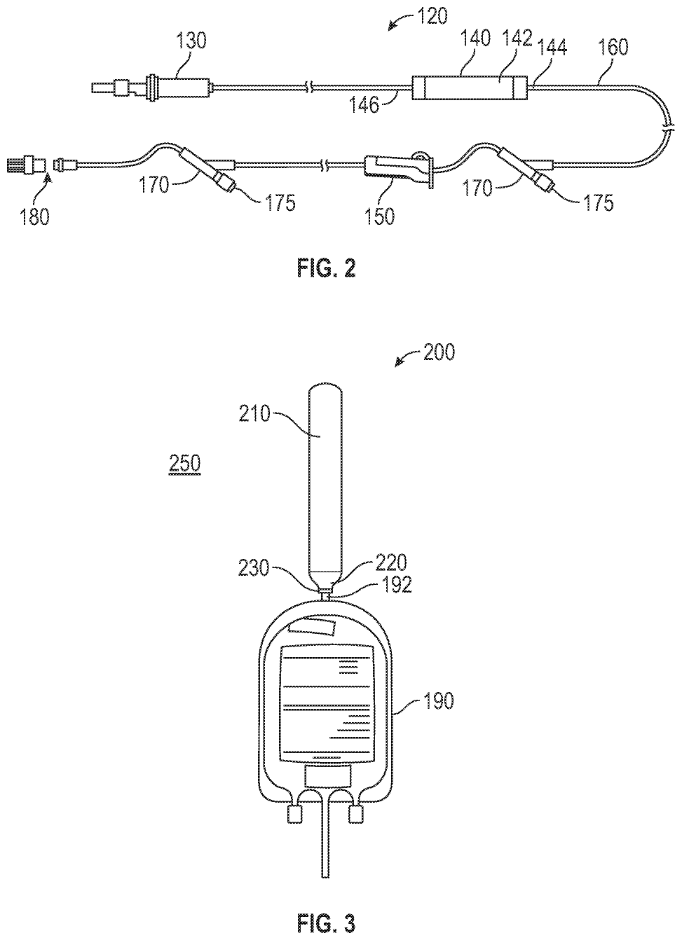

Typically, medical fluid administration sets have more parts than are shown in FIG. 1, such as those shown in FIG. 2. IV sets may be formed from any combination of infusion components and tubing. Typically, the infusion components and tubing are disposable products that are used once and then discarded. The infusion components and tubing may be formed from any suitable material (e.g., plastic, silicone, rubber), many or all of which are clear or translucent so that the fluid flow or levels inside can be seen.

As shown in FIG. 2, an IV set 120 may include a drip chamber 130, a hand pump 140 and a roller clamp 150 connected together by tubing 160. The IV set 120 may also include a Y-site 170 having a Y-shaped junction with a needleless connector 175, as well as a luer lock connector 180 at the end of the IV set 120. The luer lock connector 180 may be used for connection to a catheter inserted into a patient, for example. The IV set 120 may include additional infusion components and may be formed of any combination of components and the tubing 160.

In use, IV set 120 is connected to an IV fluid bag (e.g., blood bag) via the drip chamber 130 and the luer lock connector 180 is connected to a catheter that is placed into a vein of a patient. Thus, fluid flows from the fluid bag through the drip chamber 130 to the hand pump 140 and through the remainder of the IV set 120 and out of the luer lock connector 180. As the hand pump 140 is squeezed, the volume of fluid contained within a body 142 of the hand pump 140 is forced out an outlet check valve 144 and downstream through the IV set 120. When the hand pump 140 is released (e.g., stop squeezing), the body 142 of the hand pump 140 reinflates and draws in a new volume of fluid through an inlet check valve 146. This squeezing cycle can be repeated as often as necessary to move fluid from one or more fluid bags into the patient as quickly as possible.

The hand pump 140 is a typical flexible cylinder that is squeezed by a user continuously with one hand and released before activating or squeezing it again. However, the smooth cylindrical shape of the hand pump 140 provides a non-ergonomic shape to be gripped by a hand and no internal features to promote downstream fluid flow. Also, the squeezable cylinder shape geometry is often uncomfortable for prolonged use and may quickly lead to user hand fatigue. Further, the cylinder shape geometry is dependent on the pressure of the fluid flow coming in to the cylinder to expand it back out to a cylinder shape and fill with fluid after it has been collapsed by squeezing.

In aspects of the disclosure, a cylindrical hand pump may be augmented or replaced by a pressure assembly having a pressurized container and a connector for coupling with a fluid reservoir, such as an IV blood bag. For example, the connection may be at a top end of a hanging IV blood bag, though the connection may be at any suitable location of the fluid reservoir. The pressure assembly provides for quickly introducing pressure into the fluid reservoir in a downstream direction when openly coupled to the fluid reservoir. For example, a pressurized container of the pressure assembly may be configured so that a fluid flow pathway of the coupling may be opened (e.g., allow pressure flow from pressure assembly to fluid reservoir) upon coupling to the fluid reservoir, upon removing a seal from an already coupled pressure assembly and upon opening a valve of an already coupled pressure assembly. This decreases user hand fatigue from pumping fluid and speeds up the refill cycle in which new fluid is pushed into the body of the hand pump when augmenting the hand pump. Otherwise, this eliminates user hand fatigue from pumping fluid and speeds up the fluid flow through the IV set when replacing the hand pump.

As shown in FIG. 3, a pressure assembly 200 is provided according to aspects of the disclosure. Pressure assembly 200 may be in the form of a pressurized container including a body 210, an outlet port 220 and a connector 230. As shown, the connector 230 of the pressure assembly 200 may be coupled with a connector 192 of a fluid reservoir 190 (e.g., IV blood bag).

In aspects of the disclosure, the pressure assembly 200 may contain a pressurized gas (e.g., CO2, O2) that is suitable for use with medical fluids. In aspects of the disclosure, the pressure assembly 200 is configured to only introduce a pressure differential into the fluid reservoir 190 without introducing a gas into the medical fluid contained within the fluid reservoir 190. In aspects of the disclosure, the pressure assembly 200 may contain a pressurized fluid (e.g., saline) that is suitable for intermingling with medical fluids, which may avoid introduction of air or gas bubbles into the medical fluid.

The body 210 may be formed of any suitable material (e.g., aluminum, steel, alloy, hard plastic) that is configured to be in contact with a pressurized fluid (e.g., pressurized gas, pressurized saline). The body 210 may be sized and shaped in any suitable form, such as a cylindrical canister, for example, though the body 210 may have any suitable shape (e.g., spherical, oblong).

The connector 230 may be any suitable connector for coupling with an opposing connector 192 of a fluid reservoir 190. For example, the connector 230 may be a male Luer connector configured to mate with a connector 192 in the form of a female Luer connector, or the connector 230 may be a female Luer connector configured to mate with a connector 192 in the form of a male Luer connector. As another example, the connector 230 may be a needleless connector configured to mate with a connector 192 in the form of a needleless port, or the connector 230 may be a needleless port configured to mate with a connector 192 in the form of a needleless connector. In another example, the connector 230 may be a female snap connector configured to couple with a male snap connector of the fluid reservoir, or the connector 230 may be a male snap connector configured to mate with a connector 192 in the form of a female snap connector. In aspects of the disclosure, the connector 230 may be a spike that is configured to penetrate the fluid reservoir 190 instead of coupling to an opposing connector. In aspects of the disclosure, the outlet port 220 may be configured as the connector 230. In aspects of the disclosure, the pressure assembly 200 may only have an outlet port 220 and not a connector 230.

Thus, the interconnection between connector 230 and connector 192 may be a quick connect design that provides for a fast connection of the pressure assembly 200 when increased fluid flow is required as fast as possible. For example, a fluid flow pathway may be opened between the pressure assembly 200 and the fluid reservoir 190 upon the mating of the connector 230 to the connector 192, providing for continuous and/or constant pressure into the fluid reservoir 190 until the pressurized fluid is depleted or the connector 230 is disconnected from the connector 192.

In aspects of the disclosure, the connector 230 may have a seal covering or blocking a fluid pathway from the pressure assembly 200. For example, the pressure assembly 200 may be connected to the fluid reservoir 190 any time before the use of the pressure assembly 200 is needed. Here, once the additional pressure from the pressure assembly 200 is required, the seal may be removed, providing for continuous and/or constant pressure into the fluid reservoir 190 until the pressurized fluid is depleted, the connector 230 is disconnected from the connector 192, or the seal is put back in place.

In aspects of the disclosure, the connector 230 may have a valve disposed within a fluid pathway from the pressure assembly 200. For example, the pressure assembly 200 may be connected to the fluid reservoir 190 any time before the use of the pressure assembly 200 is needed, with the valve in a closed position. Here, once the additional pressure from the pressure assembly 200 is required, the valve may be adjusted (e.g., opened partially, opened fully), providing for continuous and/or constant pressure, for each valve setting, into the fluid reservoir 190 until the pressurized fluid is depleted, the connector 230 is disconnected from the connector 192, or the valve is adjusted back to the closed position.

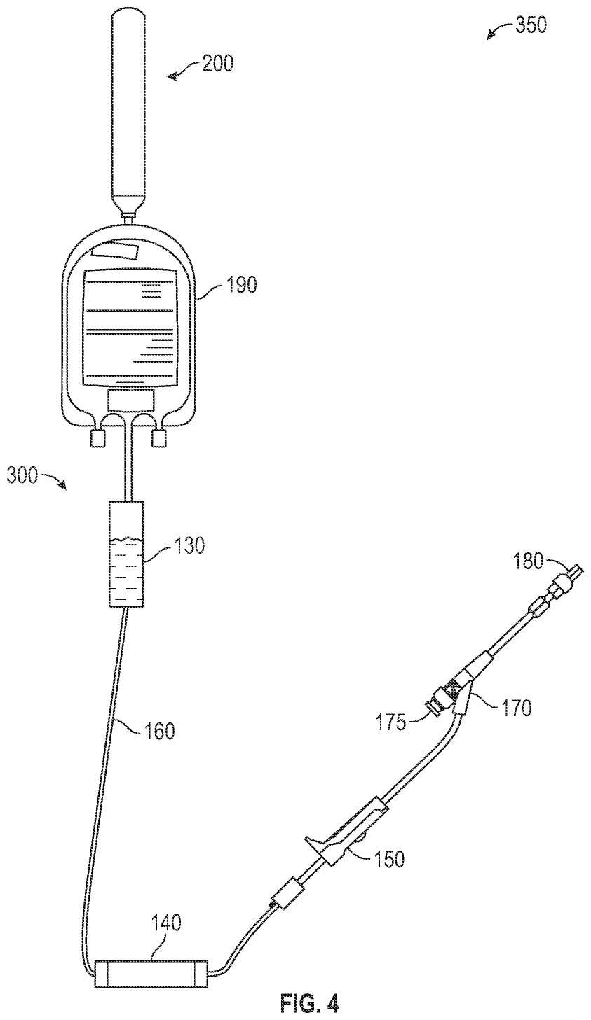

As shown in FIG. 4, the pressure assembly 200 may be part of an IV system 350. Here, the pressure assembly 200 is configured to be directly coupled to a fluid reservoir 190 (e.g., IV blood bag). The IV set 300 may include a drip chamber 130, a hand pump 140, a roller clamp 150, a Y-junction 170 having a needleless port 175 and a luer connector 180, all coupled together by IV tubing 160 and attached to the fluid reservoir 190 (e.g., to a fluid outlet of the fluid reservoir 190). Thus, an IV system 350 may be a combination of the IV set 300, including the pressure assembly 200, and the fluid reservoir 190.

In aspects of the disclosure, the pressure assembly 200 may be used in conjunction with the hand pump 140. For example, during use of the IV set 300, a user (e.g., clinician, nurse) may begin squeezing and releasing the hand pump 140 when increased fluid flow is needed, as well as open a fluid pathway between the pressure assembly 200 and the fluid reservoir 190 (e.g., remove a seal from or open a valve of the pressure assembly 200). Thus, by providing a quick burst of pressure from the pressure assembly 200 that can be added to the IV set 300 when needed (e.g., repeatedly), the pressure assembly 200 may increase the fluid flow rate and reduce the amount of times the user needs to manually squeeze the hand pump 140.

In aspects of the disclosure, the pressure assembly 200 may be used instead of the hand pump 140. For example, IV set 300 without a hand pump 140 (e.g., modified IV set 300). Here, during use of the modified IV set 300, a user (e.g., clinician, nurse) may simply open a fluid pathway between the pressure assembly 200 and the fluid reservoir 190 (e.g., remove a seal from or open a valve of the pressure assembly 200) when increased fluid flow is needed. Thus, by providing a quick burst of pressure from the pressure assembly 200 that can be added to the modified IV set 300 when needed (e.g., open once, open and shut repeatedly), the pressure assembly 200 may increase the fluid flow rate and eliminate the need for the user to manually squeeze a hand pump at all.

In aspects of the disclosure, the pressure assembly 200 may be coupled directly to a fluid reservoir 190 as a disposable reservoir combination 250 that is already coupled when the fluid reservoir 190 is put into service (e.g., an IV blood bag is hung and connected to an IV set). Here, the IV set may be all of the components of IV set 300 or modified IV set 300 excluding the pressure assembly 200. Thus, when an existing IV bag runs out for an IV set being used to deliver fluid to a patient, the depleted IV bag may be quickly replaced by a disposable reservoir combination 250 (e.g., full fluid reservoir 190 and coupled pressure assembly 200) to continue delivering fluid through the IV set.

In aspects of the disclosure, the pressure assembly 200 may be a standalone IV component that may be coupled directly to a fluid reservoir 190 after the fluid reservoir 190 is put into service (e.g., an IV blood bag is hung and connected to an IV set). Here, the IV system may be all of the components of IV system 350 or modified IV system 350 excluding the pressure assembly 200. Thus, when an existing IV bag runs out for an IV system being used to deliver fluid to a patient, the depleted IV bag may be quickly replaced by a full fluid reservoir 190 to continue delivering fluid through the IV set. Here, either the existing pressure assembly 200 may be disconnected from the depleted IV bag and connected to the full fluid reservoir 190 or a new pressure assembly 200 may be connected to the full fluid reservoir 190.

Thus, in aspects of the disclosure, the pressure assembly 200 may be a standalone component that may be used and disposed of with a single disposable fluid reservoir, used and disposed of with a disposable IV set (e.g., IV set 300), reused with multiple fluid reservoirs 190 and a single disposable IV set, or reused with multiple fluid reservoirs 190 and multiple IV sets. In aspects of the disclosure, the pressure assembly 200 may be a separate component packaged with a disposable IV set (e.g., IV set 300) where the IV set and the pressure assembly 200 are disposed of when the disposable IV set reaches its life use time limit (e.g., 24 hours, 72 hours, 7 days). In aspects of the disclosure, the pressure assembly 200 may be a separate component packaged with a fluid reservoir (e.g., IV bag 190) where the fluid reservoir and the pressure assembly 200 are disposed of when the fluid reservoir is empty and/or the pressure assembly is empty.

In aspects of the disclosure, the pressure assembly 200 may be cleaned per medical cleaning requirements and reused multiple times. For example, the pressure assembly 200 may be cleaned between each use when being switched between fluid reservoirs 190. As another example, the pressure assembly 200 may be cleaned after being depleted and then refilled with new pressurized fluid for reuse.

In use, the IV set 300 and the pressure assembly 200 are each coupled to a fluid reservoir (e.g., IV bag 190) containing a medical fluid (e.g., blood). In an unactuated state, pressure assembly 200 provides no pressure to the fluid reservoir 190, thus allowing the fluid to flow through the IV set 300 at a rate set by a flow controller (e.g., roller clamp 150). When quicker fluid flow is needed, the pressure assembly 200 may be activated (e.g., remove a seal from or open a valve of pressure assembly 200), thus increasing pressure into the fluid reservoir 190 and forcing the fluid out of the fluid reservoir 190 more quickly. In aspects of the disclosure, the unactuated state is achieved by the pressure assembly 200 not being coupled to the fluid reservoir 190 and the pressure assembly 200 is only coupled to the fluid reservoir 190 when activation of the pressure assembly 200 is desired. Here, the increased pressure into the fluid reservoir 190 occurs upon coupling of the pressure assembly 200 to the fluid reservoir 190.

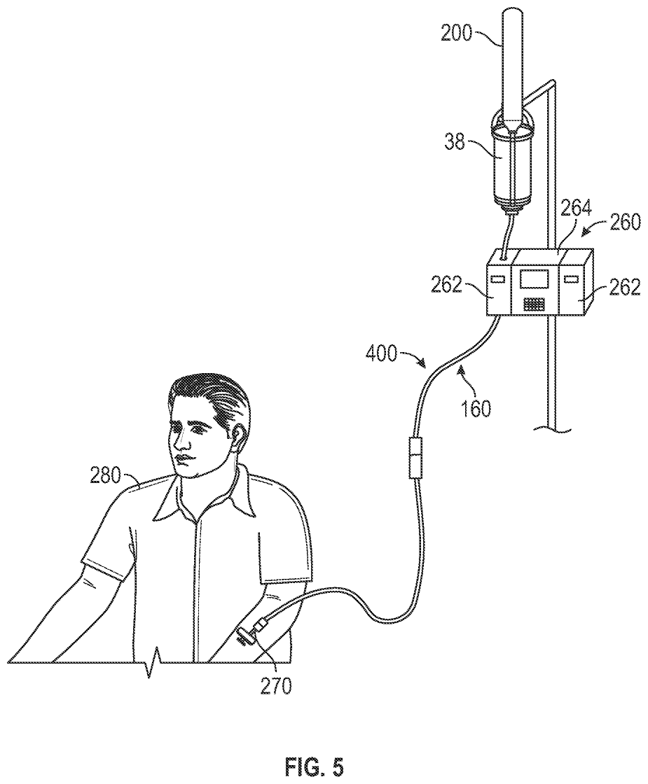

As shown in FIG. 5, an IV set 400 and a pressure assembly 200 may be coupled to a fluid source 38, with the IV set 400 being coupled to an infusion pump 260 system having two infusion pumps 262 and a controller 264, and to a catheter 270 inserted into a patient 280. Here, if the maximum fluid flow rate from the infusion pump system 260 is not sufficient, the pressure assembly 200 may be activated (e.g., connected or opened) to force the fluid to flow out of the fluid source 38 more quickly. For example, the IV set 400 may be quickly uncoupled from the infusion pump system 260 before using the pressure assembly 200 and/or the infusion pump system 260 may be set to neutral to allow unimpeded fluid flow from the fluid source 38 before using the pressure assembly 200.

In aspects of the disclosure, the pressure assembly 200 may include any suitable fastener to attach the pressure assembly 200 to an IV pole 46, a bed, an operating table, a fluid reservoir and the like. For example, the fastener may be a cradle, a hangar, a hook, Velcro ®, adhesive, and/or any other suitable fastener. In aspects of the disclosure, the pressure assembly 200 may be configured to simply extend from the fluid reservoir without being attached to anything else.

In aspects of the disclosure, the geometry and/or shape of the body 210 may be varied based on hand size and/or desired flow volume. For example, there may be small, medium and large sizes for use by different sized users. In aspects of the disclosure, the flow rates may be configured based on size/shape of the body 210 and diameter of the outlet port 220.

In aspects of the disclosure, drugs may be administered to a patient quickly via a pressure assembly 200 that forces the drugs via the IV set 300 at a rate based on the pressure provided by the pressure assembly 200.

In one or more embodiments, an IV pressure assembly comprises: a body; an outlet port disposed on the body; and a connector coupled to the outlet port, the connector configured to couple with a fluid reservoir, wherein the IV pressure assembly is configured as a pressurized container.

In aspects of the disclosure, the pressurized container contains a pressurized gas suitable for intermingling with a medical fluid within the fluid reservoir. In aspects of the disclosure, the pressurized container is configured to introduce a pressure differential into the fluid reservoir without introducing a gas into medical fluid contained within the fluid reservoir. In aspects of the disclosure, the pressurized container contains a pressurized fluid that is suitable for intermingling with a medical fluid within the fluid reservoir. In aspects of the disclosure, the connector is one of: a male luer connector configured to couple with a female luer connector of the fluid reservoir; and a female luer connector configured to couple with a male luer connector of the fluid reservoir. In aspects of the disclosure, the connector is one of: a female snap connector configured to couple with a male snap connector of the fluid reservoir; and a male snap connector configured to couple with a female snap connector of the fluid reservoir. In aspects of the disclosure, the connector is one of: a needleless connector configured to couple with a needleless port of the fluid reservoir; and a needleless port configured to couple with a needleless connector of the fluid reservoir. In aspects of the disclosure, the connector is a spike configured to penetrate the fluid reservoir instead of coupling to an opposing connector. In aspects of the disclosure, the outlet port is configured as the connector.

In aspects of the disclosure, the pressurized container is configured to open a fluid flow pathway between the IV pressure assembly and the fluid reservoir upon coupling of the connector to the fluid reservoir. In aspects of the disclosure, the pressurized container is configured to provide pressure into the fluid reservoir through the fluid flow pathway until one of: a pressurized fluid in the pressurized container is depleted; and the connector is uncoupled from the fluid reservoir. In aspects of the disclosure, the pressurized container comprises a valve configured to block a fluid flow pathway between the IV pressure assembly and the fluid reservoir when the connector is coupled to the fluid reservoir and the valve is in a closed position. In aspects of the disclosure, a seal is configured to block a fluid flow pathway between the IV pressure assembly and the fluid reservoir when the connector is coupled to the fluid reservoir. In aspects of the disclosure, the pressurized container is configured to provide pressure into the fluid reservoir through the fluid flow pathway when the seal is removed until one of: the seal is put back into a blocking position; a pressurized fluid in the pressurized container is depleted; and the connector is uncoupled from the fluid reservoir. In aspects of the disclosure, a valve is configured to block a fluid flow pathway between the IV pressure assembly and the fluid reservoir when the connector is coupled to the fluid reservoir and the valve is in a closed position. In aspects of the disclosure, the pressurized container is configured to provide pressure into the fluid reservoir through the fluid flow pathway when the valve is adjusted to one of a partially open position and a fully open position until one of: the valve is adjusted back to a closed position; a pressurized fluid in the pressurized container is depleted; and the connector is uncoupled from the fluid reservoir.

In one or more embodiments, an IV system comprises: an IV set comprising: one or more IV components; and one or more IV tubes; and an IV pressure assembly, comprising: a body; an outlet port disposed on the body; and a connector coupled to the outlet port, the connector configured to couple with a fluid reservoir, wherein the IV pressure assembly is a pressurized container configured to force fluid within the fluid reservoir into the IV set at a fluid flow rate exceeding one of a maximum gravity based fluid flow rate and a maximum infusion pump fluid flow rate.

In one or more embodiments, a method of operating an intravenous (IV) pressure assembly comprises: coupling the IV pressure assembly to a fluid reservoir, the IV pressure assembly comprising: a body comprising a pressurized container; an outlet port disposed on the body; and a connector coupled to the outlet port, the connector configured to couple with the fluid reservoir; opening a fluid flow pathway between the IV pressure assembly and the fluid reservoir; providing pressure into the fluid reservoir through the fluid flow pathway; and ceasing providing pressure into the fluid reservoir by one of: depleting a pressurized fluid in the pressurized container; uncoupling the connector from the fluid reservoir; adjusting a valve of the IV pressure assembly to a closed position; and placing a seal into a blocking position within the fluid flow pathway.

In aspects of the disclosure, wherein prior to opening the fluid flow pathway between the IV pressure assembly and the fluid reservoir, the method further comprises: flowing fluid through an IV set coupled to the fluid reservoir at a gravity based fluid flow rate; operating the IV pressure assembly to increase the fluid flow rate to be greater than one of: a maximum gravity based fluid flow rate of the IV set; a greater than maximum gravity based fluid flow rate based on operation of a hand pump of the IV set; and a maximum infusion pump fluid flow rate based on an infusion pump coupled to the IV set. In aspects of the disclosure, the method further comprises reducing user hand fatigue and increasing a fluid flow rate to reduce fluid transfusion time by one of: augmenting a hand pump of an IV set coupled to the fluid reservoir; and replacing a hand pump of an IV set coupled to the fluid reservoir.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, inserting and the like via a hardware element.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous (IV) pressure assembly comprising:
a body;
an outlet port disposed on the body; and
a connector coupled to the outlet port, the connector configured to couple with a fluid reservoir,
wherein the IV pressure assembly is configured as a pressurized container.

2. The IV pressure assembly of claim 1, wherein the pressurized container contains a pressurized gas suitable for intermingling with a medical fluid within the fluid reservoir.

3. The IV pressure assembly of claim 1, wherein the pressurized container is configured to introduce a pressure differential into the fluid reservoir without introducing a gas into medical fluid contained within the fluid reservoir.

4. The IV pressure assembly of claim 1, wherein the pressurized container contains a pressurized fluid that is suitable for intermingling with a medical fluid within the fluid reservoir.

5. The IV pressure assembly of claim 1, wherein the connector is one of:
a male luer connector configured to couple with a female luer connector of the fluid reservoir; and
a female luer connector configured to couple with a male luer connector of the fluid reservoir.

6. The IV pressure assembly of claim 1, wherein the connector is one of:
a female snap connector configured to couple with a male snap connector of the fluid reservoir; and
a male snap connector configured to couple with a female snap connector of the fluid reservoir.

7. The IV pressure assembly of claim 1, wherein the connector is one of:
a needleless connector configured to couple with a needleless port of the fluid reservoir; and
a needleless port configured to couple with a needleless connector of the fluid reservoir.

8. The IV pressure assembly of claim 1, wherein the connector is a spike configured to penetrate the fluid reservoir instead of coupling to an opposing connector.

9. The IV pressure assembly of claim 1, wherein the outlet port is configured as the connector.

10. The IV pressure assembly of claim 1, wherein the pressurized container is configured to open a fluid flow pathway between the IV pressure assembly and the fluid reservoir upon coupling of the connector to the fluid reservoir.

11. The IV pressure assembly of claim 10, wherein the pressurized container is configured to provide pressure into the fluid reservoir through the fluid flow pathway until one of:
a pressurized fluid in the pressurized container is depleted; and
the connector is uncoupled from the fluid reservoir.

12. The IV pressure assembly of claim 10, further comprising a valve configured to block a fluid flow pathway between the IV pressure assembly and the fluid reservoir when the connector is coupled to the fluid reservoir and the valve is in a closed position.

13. The IV pressure assembly of claim 1, further comprising a seal configured to block a fluid flow pathway between the IV pressure assembly and the fluid reservoir when the connector is coupled to the fluid reservoir.

14. The IV pressure assembly of claim 13, wherein the pressurized container is configured to provide pressure into the fluid reservoir through the fluid flow pathway when the seal is removed until one of:
the seal is put back into a blocking position;
a pressurized fluid in the pressurized container is depleted; and
the connector is uncoupled from the fluid reservoir.

15. The IV pressure assembly of claim 1, further comprising a valve configured to block a fluid flow pathway between the IV pressure assembly and the fluid reservoir when the connector is coupled to the fluid reservoir and the valve is in a closed position.

16. The IV pressure assembly of claim 15, wherein the pressurized container is configured to provide pressure into the fluid reservoir through the fluid flow pathway when the valve is adjusted to one of a partially open position and a fully open position until one of:
the valve is adjusted back to a closed position;
a pressurized fluid in the pressurized container is depleted; and
the connector is uncoupled from the fluid reservoir.

17. An intravenous (IV) system comprising:
an IV set comprising:
one or more IV components; and
one or more IV tubes; and
an IV pressure assembly, comprising:
a body;
an outlet port disposed on the body; and
a connector coupled to the outlet port, the connector configured to couple with a fluid reservoir,
wherein the IV pressure assembly is a pressurized container configured to force fluid within the fluid reservoir into the IV set at a fluid flow rate exceeding one of a maximum gravity based fluid flow rate and a maximum infusion pump fluid flow rate.

18. A method of operating an intravenous (IV) pressure assembly, the method comprising:
coupling the IV pressure assembly to a fluid reservoir, the IV pressure assembly comprising:
a body comprising a pressurized container;
an outlet port disposed on the body; and
a connector coupled to the outlet port, the connector configured to couple with the fluid reservoir;
opening a fluid flow pathway between the IV pressure assembly and the fluid reservoir;
providing pressure into the fluid reservoir through the fluid flow pathway; and
ceasing providing pressure into the fluid reservoir by one of:
depleting a pressurized fluid in the pressurized container;
uncoupling the connector from the fluid reservoir;
adjusting a valve of the IV pressure assembly to a closed position; and
placing a seal into a blocking position within the fluid flow pathway.

19. The method of claim 18, wherein prior to opening the fluid flow pathway between the IV pressure assembly and the fluid reservoir, the method further comprising:
flowing fluid through an IV set coupled to the fluid reservoir at a gravity based fluid flow rate;
operating the IV pressure assembly to increase the fluid flow rate to be greater than one of:
a maximum gravity based fluid flow rate of the IV set;
a greater than maximum gravity based fluid flow rate based on operation of a hand pump of the IV set; and
a maximum infusion pump fluid flow rate based on an infusion pump coupled to the IV set.

20. The method of claim 18, further comprising:
reducing user hand fatigue and increasing a fluid flow rate to reduce fluid transfusion time by one of:
augmenting a hand pump of an IV set coupled to the fluid reservoir; and
replacing a hand pump of an IV set coupled to the fluid reservoir.
